(19) United States
(12) Patent Application Publication
(10) Pub. No.: US 2012/0010821 A1
(43) Pub. Date: Jan. 12, 2012

(54) Nucleic Acid Abundance Ratio Measurement Device, Method, and Program Storage Medium, Determination Method and Nucleic Acid Abundance Ratio Measurement Kit

(75) Inventors: Katsuyasu Hirano, Kyoto-shi (JP); Mariko Komori, Kyoto-shi (JP)

(73) Assignee: ARKRAY, Inc., Kyoto (JP)

(21) Appl. No.: 13/177,394

(22) Filed: Jul. 6, 2011

Related U.S. Application Data
(60) Provisional application No. 61/362,366, filed on Jul. 8, 2010.

(30) Foreign Application Priority Data
Jul. 7, 2010 (JP) 2010-155032
Jul. 4, 2011 (JP) 2011-148520

(51) Int. Cl. G06F 19/00; C12Q 1/68

(52) U.S. Cl. 702/19; 435/6.11

(57) ABSTRACT

A nucleic acid abundance ratio measurement device includes a detection section that detects a detection signal over different temperature ranges of a melting curve for a nucleic acid mixture having one or more melting temperatures, and an abundance ratio computation section that computes a nucleic acid abundance ratio based on a ratio of characteristic amounts obtained from the detection signal detected by the detection section and based on detection amount data.

### CROSS-REFERENCE TO RELATED APPLICATION

This application claims priority under 35 USC 119 from Japanese Patent Applications No. 2010-155032 filed on Jul. 7, 2010, No. 2011-148520 filed on July 4, 2011, and U.S. Provisional Application No. 61/362,366 filed on Jul. 8, 2010, the disclosures of are incorporated by reference herein.

### BACKGROUND

### Field of the Invention

The present invention relates to a nucleic acid abundance ratio measurement device, a nucleic acid abundance ratio measurement method, a nucleic acid abundance ratio measurement program storage medium, a determination method and a nucleic acid abundance ratio measurement kit.

A proposal has been made for a melting curve analysis method and a melting curve analyzing device for discriminating the type of a polymorphous genes by determining the presence or absence of two different peaks and their temperature ranges from a melting curve expressed by a differential melting curve that expresses a relationship between a signal differential value, which is a differential of a signal value of a melting curve expressing signal values of a sample in a range of temperatures, and temperature (see, for example, International Publication (WO) No. 2009/081965).

However, while the proposal of WO 2009/081965 is capable of automatically discriminating the type of a polymorphous gene, it is not capable of obtaining the proportion of the gene that is mutated.

### SUMMARY

In consideration of the above circumstances, the present invention provides a nucleic acid abundance ratio measurement device, a nucleic acid abundance ratio measurement method, a nucleic acid abundance ratio measurement program storage medium, a determination method and a nucleic acid abundance ratio measurement kit that are capable of measuring a nucleic acid abundance ratio by easily computing a nucleic acid abundance ratio.

A first aspect of the present invention is a nucleic acid abundance ratio measurement device including: a detection section that detects a detection signal over different temperature ranges of a melting curve for a nucleic acid mixture having one or more melting temperatures; and an abundance ratio computation section that computes a nucleic acid abundance ratio based on a ratio of characteristic amounts obtained from the detection signal detected by the detection section and based on detection amount data. The detection amount data is data expressing a relationship between ratios of characteristic amounts and nucleic acid abundance ratios, and is, for example a detection amount curve, table, or computation formula expressing the relationship between the ratios of characteristic amounts and the nucleic acid abundance ratios.

According to the first aspect, the detection section detects a detection signal over different temperature ranges of a melting curve foe a nucleic acid mixture having one or more melting temperatures. Then the abundance ratio computation section measures a nucleic acid abundance ratio in a sample (a nucleic acid mixture to be measured) by computing a nucleic acid abundance ratio based on a ratio of characteristic amounts obtained from the detection signal detected by the detection section and the detection amount data.

The first aspect may further include: a storage section that stores the detection amount data that, based on respective melting curves each expressing a relationship between temperature and a detection signal obtained from each of a plurality of nucleic acid mixtures of different abundance ratios of nucleic acids having different melting temperatures, expresses a relationship between ratios of characteristic amounts obtained from the detection signal of the melting curves over a plurality of temperature ranges respectively containing the melting temperatures, and the abundance ratios; or an input section that inputs the detection amount data.

According to this configuration, the storage section stores detection amount data that, based on respective melting curves each expressing the relationship between temperature and detection signal obtained from each of plural nucleic acid mixtures of different abundance ratios of nucleic acids having different respective melting temperatures, expresses a relationship between ratios of characteristic amounts obtained from the detection signal of the melting curves over plural temperature ranges respectively containing the melting temperatures, and the abundance ratios. Or the detection amount data is input via the input section. The nucleic acid abundance ratio of a sample is measured by computing the nucleic acid abundance ratio based on the detection amount data and the ratio of characteristic amounts, which is computed based on the detection signals over the respective plural temperature ranges of the melting curve of the nucleic acid mixture to be measured (measurement target).

Accordingly, the nucleic acid abundance ratio in a sample can be measured by simple computation based on the detection amount data expressing the relationship between plural abundance ratios and the ratio of characteristic amounts obtained from the detection signal of the melting curves over the plural temperature ranges containing the melting temperatures of the respective nucleic acids, and the ratio of characteristic amounts obtained from the detection signal of the melting curve over the plural temperature ranges of the measurement target nucleic acid mixture.

In the first aspect, the characteristic amount ratio may be set as one of: a ratio of a first level of the detection signal level (substantially at) a first melting temperature from which a background level thereof has been subtracted, and a second level of the detection signal near to a second melting temperature from which a background level thereof has been subtracted; a ratio of a first differential value of the detection signal level near to a first melting temperature from which a background level thereof has been subtracted and a second differential value of the detection signal level near to a second melting temperature from which a background level thereof has been subtracted; a ratio of a first sum of differential values of the detection signal over a first temperature range and a second sum of differential values of the detection signal over a second temperature range; or a ratio of a first sum of differential values of the detection signal over a first temperature range from which a sum of background levels thereof has been subtracted, and a second sum of differential values of the detection signal over a second temperature range from which a sum of background levels thereof has been subtracted.

The level of the detection signal level near to (substantially at) a melting temperature means that while it is preferable to employ a detection signal at the melting temperature, a detection signal near to the melting temperature is employed in consideration of errors arising due to fluctuations in melting temperature occurring due to test conditions (the state of the sample) and cycle number, and the detection sensitivity (for example measurement in 1° C. units) of measurement devices. The detection signal at the melting temperature is included in the detection signal near to the melting temperature.

In the first aspect, the characteristic amount ratio may be set as a ratio, in a second melting curve expressing a relationship between temperature and differential values of the detection signal, of a first surface area of a region bounded by a straight line passing through a point corresponding to a lower limit value of a first temperature range and a point corresponding to an upper limit value of the first temperature range and bounded by the melting curve, and a second surface area of a region bounded by a straight line passing through a point corresponding to a lower limit value of a second temperature range and a point corresponding to an upper limit value of the second temperature range and bounded by the melting curve.

The lower limit values or the upper limit values of the first and second temperature ranges can be selected from: temperatures at which the differential value takes a minimum value between two peaks of the second melting curve, temperatures corresponding to tails of the peak of the second melting curve, or temperatures within ±15° C. from a temperature corresponding to the peak of the second melting curve.

Or, the lower limit values or the upper limit values of the first and second temperature ranges may be temperatures within ±10° C. from a temperature corresponding to the peak of the second melting curve.

Further alternatively, the lower limit values or the upper limit values of the first and second temperature ranges may be temperatures within ±7° C. from a temperature corresponding to the peak of the second melting curve.

In these configurations, the upper limit value of one of the first or second temperature range may be different from the lower limit value of the other of the first or second temperature range that is a higher range than that of the one of the first or second temperature range.

A width from the lower limit value to the upper limit value of the first temperature range and a width from the lower limit value to the upper limit value of the second temperature range may be identical.

Or, a width from the lower limit value to the upper limit value of the first temperature range and a width from the lower limit value to the upper limit value of the second temperature range may be different.

The first aspect may further include: a temperature controller that controls change in temperature of the nucleic acid mixture; and a measurement section that measures degree of light absorption, fluorescence intensity or relative fluorescence intensity as the detection signal. By adopting such a configuration the measured detection signal from a sample can be employed to determine the nucleic acid abundance ratio in the sample.

A second aspect of the present invention is a nucleic acid abundance ratio measurement method including: detecting a detection signal over different temperature ranges for a melting curve of a nucleic acid mixture having one or more melting temperatures; and computing a nucleic acid abundance ratio based on a ratio of characteristic amounts obtained from the detection signal that has been detected and based on detection amount data.

The above nucleic acid abundance ratio measurement method detects a detection signal over different temperature ranges for a melting curve of a nucleic acid mixture having one or more melting temperatures. Then a nucleic acid abundance ratio in a sample is measured by computing the nucleic acid abundance ratio based on a ratio of characteristic amounts obtained from the detection signal that has been detected and based on detection amount data.

A third aspect of the present invention is a nucleic acid abundance ratio measurement method including: generating detection amount data based on melting curves each expressing a relationship between temperature and a detection signal obtained from each of a plurality of nucleic acid mixtures of different abundance ratios of nucleic acids having different respective melting temperatures, the detection amount data expressing a relationship between ratios of characteristic amounts obtained from the detection signal of the melting curves over a plurality of temperature ranges respectively containing the melting temperatures, and the abundance ratios; computing a ratio of characteristic amounts based on a detection signal of a melting curve of a measurement target nucleic acid mixture over temperature ranges corresponding to each of the plurality of respective temperature ranges; and computing a nucleic acid abundance ratio based on the computed ratio of characteristic amounts and the generated detection amount data. The nucleic acid abundance ratio can be measured by computing the nucleic acid abundance ratio through these processes

A fourth aspect of the present invention is a non-transitory storage medium storing a program that causes a computer to execute abundance ratio measurement processing, the abundance ratio measurement processing including: receiving a detection signal of a melting curve of a nucleic acid mixture having one or more melting temperatures, the detection signal being detected over different temperature ranges; and computing a nucleic acid abundance ratio based on a ratio of characteristic amounts obtained from the received detection signal and based on detection amount data.

A fifth aspect of the present invention is a non-transitory storage medium storing a program that causes a computer to execute abundance ratio measurement processing, the abundance ratio measurement processing including: acquiring detection amount data from a storage section that stores the detection amount data that, based on respective melting curves each expressing a relationship between temperature and a detection signal obtained for each of a plurality of nucleic acid mixtures of different abundance ratios of nucleic acids having different respective melting temperatures, expresses a relationship between ratios of characteristic amounts obtained from the detection signals of the melting curves over a plurality of temperature ranges respectively containing the melting temperatures, and respective abundance ratios, or acquiring the detection amount data input by an input section; computing a ratio of characteristic amounts based on a detection signal of a melting curve of a measurement target nucleic acid mixture, the detection signal detected over temperature ranges respectively corresponding to each of the plurality of temperature ranges; and computing a nucleic acid abundance ratio based on the computed ratio of characteristic amounts and the acquired detection amount data. A computer is accordingly able to measure the abundance ratio of nucleic acids in a test sample. An alternative aspect is a nucleic acid abundance ratio measurement program that causes the computer to function as a controller that controls another computer to perform computation of the detection signal or a portion of the computation.

A sixth aspect of the present invention is a determination method of determining a condition of a patient based on: a nucleic acid abundance ratio obtained with any one of the preceding aspects; and a relationship determined in advance between nucleic acid abundance ratios and patient condition.

A seventh aspect of the present invention is a determination method of determining a constitution of a patient and/or an appropriate drug administration amount for the constitution based on: a nucleic acid abundance ratio obtained with any one of the preceding aspects; and a relationship determined in advance between nucleic acid abundance ratios and constitutions and/or appropriate drug administration amount for the constitutions.

An eighth aspect of the present invention is a nucleic acid abundance ratio measurement kit that is used in the measurement of a nucleic acid abundance ratio with any one of the preceding aspects, the kit including: a probe that can be hybridized with a region of a nucleic acid sequence that includes a target mutation that may exist in the nucleic acid mixture; and a set of primers that can amplify the nucleic acid sequence that includes the target mutation. The nucleic acid abundance ratio measurement kit can be also used for determining a constitution and/or an appropriate drug administration amount for the constitution based on the relationship between the predetermined nucleic acid abundance ratio and the constitution and/or the appropriate drug administration amount for the constitution.

Other aspect of the present invention is a nucleic acid abundance ratio measurement device that includes: a storage section that stores detection amount data that, based on respective melting curves expressing the relationship between temperature and detection signal obtained from each of plural nucleic acid mixtures of different abundance ratios of nucleic acids in plural respective abundance ratios, expresses a relationship between ratios of characteristic amounts obtained from the detection signal of the melting curves over plural temperature ranges containing respective different melting temperatures and the abundance ratios; a characteristic amount ratio computation section that computes a ratio of characteristic amounts based on a detection signal of a melting curve of a measurement target nucleic acid mixture over temperature ranges corresponding to the plural respective temperature ranges; and a nucleic acid abundance ratio computation section that computes a nucleic acid abundance ratio based on the ratio characteristic amounts computed by the characteristic amount ratio computation section and the detection amount data stored in the storage section.

According to the above aspects for measuring abundance ratio of nucleic acids as explained above, an abundance ratio of nucleic acids in a sample can be measured easily by computing the nucleic acid abundance ratio based on a ratio of characteristic amounts obtained from a detection signal of a melting curve of a mixture of nucleic acids having different melting temperatures and based on detection amount data.

According to the aspects of determination method and kit, the nucleic acid abundance ratio obtained by any one of the aspects for measuring abundance ratio of nucleic acids can be employed to determine the condition of a patient.

### BRIEF DESCRIPTION OF THE DRAWINGS

Exemplary embodiments of the present invention will be described in detail based on the following figures, wherein:

FIG. 1 is a block diagram illustrating a configuration of a nucleic acid abundance ratio determination device of an exemplary embodiment;

FIG. 2A is a graph illustrating an example of a melting curve of a nucleic acid mixture, and FIG. 2B is a graph illustrating a differential melting curve of the example;

FIG. 3 is a graph illustrating another example of a differential melting curve;

FIG. 4 is a graph illustrating an example of a detection amount curve;

FIG. 5 is a flow chart illustrating the contents of a nucleic acid abundance ratio measurement routine for a nucleic acid abundance ratio determination device of the exemplary embodiment;

FIG. 6 is a graph illustrating differential melting curves for each sample of nucleic acid mixture in an Example;

FIG. 7 is a graph illustrating a melting curve of a nucleic acid mixture to be measured (measurement target) in the Example;

FIG. 8 is a graph illustrating a differential melting curve of the measurement target nucleic acid mixture in the Example;

FIG. 9A is a graph illustrating another example of a melting curve of a nucleic acid mixture, and FIG. 9B is a graph illustrating a differential melting curve of the another example;

FIG. 10A and FIG. 10B are graphs for explaining another example of a ratio of characteristic amounts;

FIG. 11 is a diagram illustrating an example of a differential of a melting curve in which two temperature ranges are separated from each other; and

FIG. 12 is a graph illustrating an example of a differential melting curve in which there is a significant difference in base values.

### DETAILED DESCRIPTION

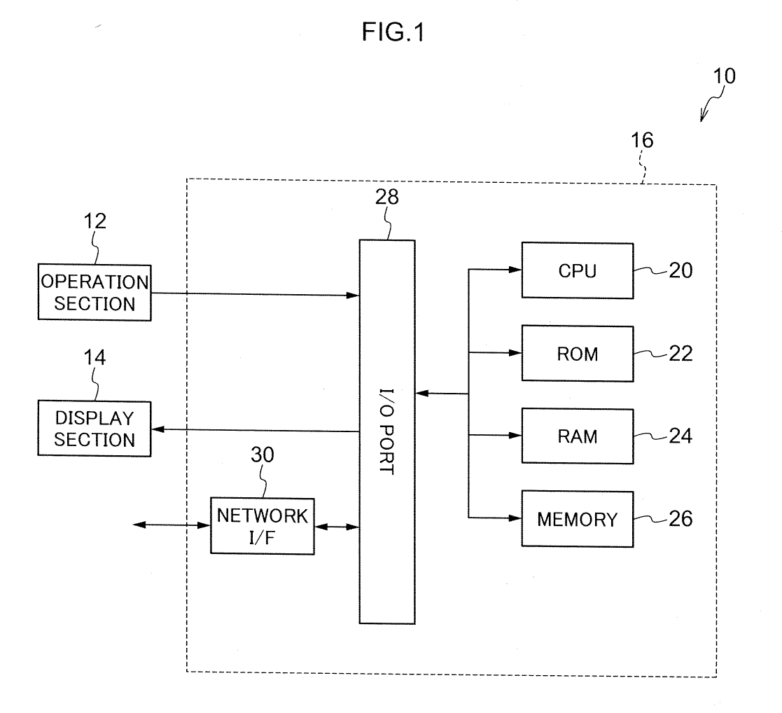

As shown in FIG. 1, a nucleic acid abundance ratio measurement device 10 of an exemplary embodiment includes: an operation section 12 configured with a keyboard, mouse, touch panel and/or a reader, such as barcode reader, and operated to input various types of data; a display section 14 that displays results of nucleic acid abundance ratio measurement; and a computer 16 that executes nucleic acid abundance ratio measurement processing.

The computer 16 includes: a CPU 20 that controls the nucleic acid abundance ratio measurement device 10 overall; ROM 22 that serves as a storage medium that stores various programs, including a program for nucleic acid abundance ratio measurement processing as described later; RAM 24 that serves as a working area for temporary storage of data; a memory 26 that serves as a storage section in which various data is stored; an input-output port (I/O port) 28; a network interface (network I/F) 30; and a bus that connects these sections together. The operation section 12 and the display section 14 are connected to the I/O port 28. A HDD may also be provided to the computer 16.

The memory 26 is stored with a detection amount curve that, based on melting curves for plural nucleic acid mixtures having different abundance ratios of two types of nucleic acid, expresses the relationship between, over two temperature ranges that include different melting temperatures, a ratio of the sum of differential values of a detection signal for a melting curve from which the sum of background levels thereof has been subtracted, and the abundance ratio of the two types of nucleic acid. The detection amount curve may be stored in the ROM 22 or in a HDD. In the present exemplary embodiment, explanation is given of a case in which stored detection amount curves are read out prior to computation. However configuration may be made such that detection amount curve data is input to the operation section 12 by, for example, reading in a bar code including data for the detection amount curves with a bar code reader, reading in the detection amount curve data stored on an IC chip or RFID with various readers, or receiving the detection amount curve from an external device connected through the I/O port 28 and the network I/F 30.

Explanation follows regarding a detection amount curve generation method.

First, for example, plural nucleic acid mixtures are prepared that each have different abundance ratios of two types of nucleic acid, a wild-type nucleic acid Wt and a mutant nucleic acid Mt. Melting curves are obtained with a melting curve analysis instrument for each of the plural nucleic acid mixtures.

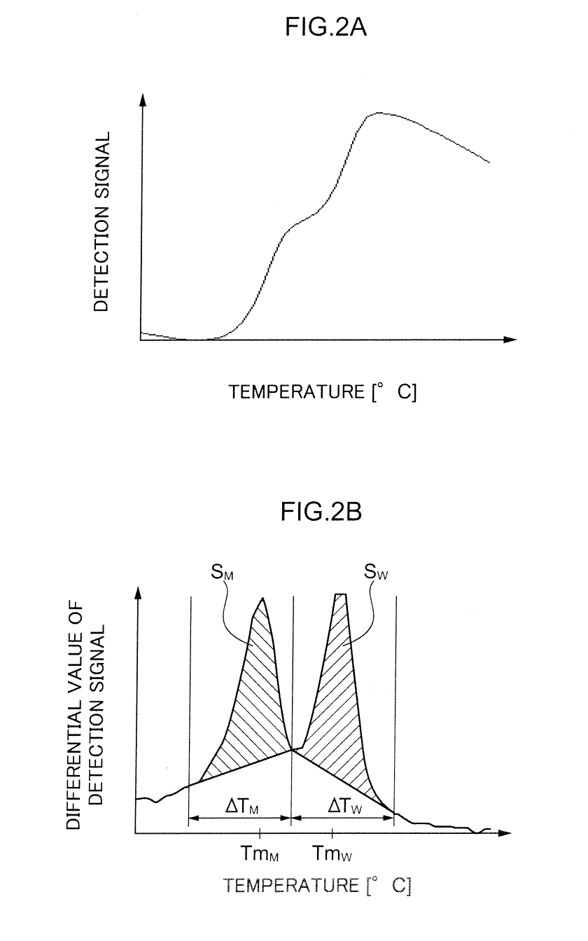

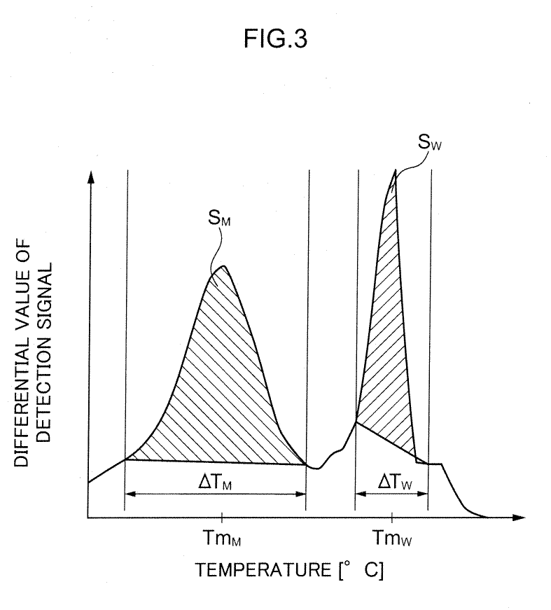

FIG. 2A illustrates a melting curve expressing the relationship for a single nucleic acid mixture of a detection signal, such as a degree of light absorption or fluorescence intensity, to temperature. FIG. 2B illustrates a melting curve (also called a differential melting curve, which corresponds to the second melting curve of the present invention) expressing the relationship of the differential values of the detection signal to temperature. The melting temperature Tm_{W} of the nucleic acid Wt and the melting temperature Tm_{M} of the mutant nucleic acid Mt are detected from the peaks of the differential melting curve, and temperature ranges that contain Tm_{W} and Tm_{M}, respectively, are set. A temperature range ΔT_{W} containing Tm_{M} can be set, for example, with a lower limit at the temperature at which the differential value of the detection signal reaches a minimum between Tm_{W} and Tm_{M}, and with an upper limit at the temperature corresponding to the tail of the peak of the detection signal. A temperature range ΔT_{M} containing Tm_{M} can be set, for example, with an upper limit at the temperature at which the differential value of the detection signal reaches a minimum between Tm_{W} and Tm_{M}, and with a lower limit at a temperature corresponding to the tail of the peak of the detection signal The temperature range ΔT_{W} and the temperature range ΔT_{M} can be set so as to have the same width as each other (for example 10° C.) as shown in FIG. 2B, or set to have different widths from each other (for example a temperature range T_{W} of 10°, and a temperature range T_{M} of 7° C.) as shown in FIG. 3. Further, the temperature range ΔT_{W} and the temperature range ΔT_{M} can be set with widths from minus X° C. to plus X° C. from the melting temperatures Tm_{W} and Tm_{M}, respectively, (X may be for example 15° C. or less, preferably 10° C. or less, and more preferably 7° C. or less). Such setting of temperature ranges can be easily implemented automatically.

Then, for each of the temperature range ΔT_{W} and the temperature range ΔT_{M}, a surface area is derived of an area bounded by a straight line passing through a point corresponding to the lower limit and a point corresponding to the upper limit of the respective temperature range of the differential melting curve and bounded by the differential melting curve itself (i.e., the shaded regions in FIG. 2B). A specific example for a method that can be employed for deriving the surface area is set out below. The surface area S can be derived from the following Equation (1), wherein f (T) is a differential value of the detection signal at temperature T, and B (T) is a base value at temperature T.

Surface Area S={f(Tₛ₊₁)−B(Tₛ₊₁)}+{f(T ₛ₊₂)−B(T ₛ₊₂)} and so on up to {f(Tₑ₋₁)−B(Tₑ₋₁)} Equation (1)

wherein is the lower limit value of each of the temperature ranges. and Tₑ is the upper limit value thereof. The base value B (T) at each temperature T is a value derived according to the following Equation (2), and represents the background level included in the detection signal. Influence from background included in the detection signal can be appropriately removed by subtracting this base value from the differential value of the detection signal.

B(T)=a×(T−Tₛ)+f(Tₛ) wherein a={f(Tₑ)−f(Tₛ)}/(Tₑ−Tₛ). Equation (2)

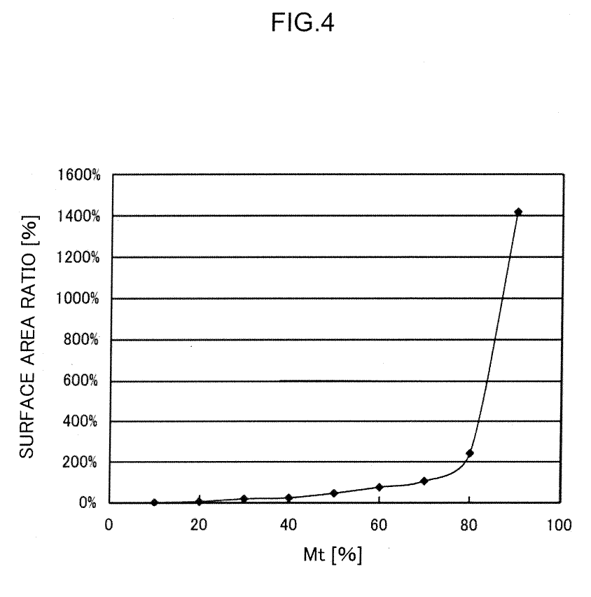

For each nucleic acid mixture the surface area S_{W} over the temperature range ΔT_{W} and the surface area S_{M} over the temperature range ΔT_{M} are derived according to Equation (1) and Equation (2). A detection amount curve is then generated that expresses the relationship between the surface area ratios and the abundance ratios for each of the nucleic acid mixtures. FIG. 4 illustrates an example of a detection amount curve, with the abundance ratio (the proportion of nucleic acid Mt to the total nucleic acid mixture) on the horizontal axis and the surface area ratio (S_{M}/S_{W}) on the vertical axis. The thus generated detection amount curve is stored in the memory 26. The surface area ratio may also be defined as (S_{W}/S_{M}).

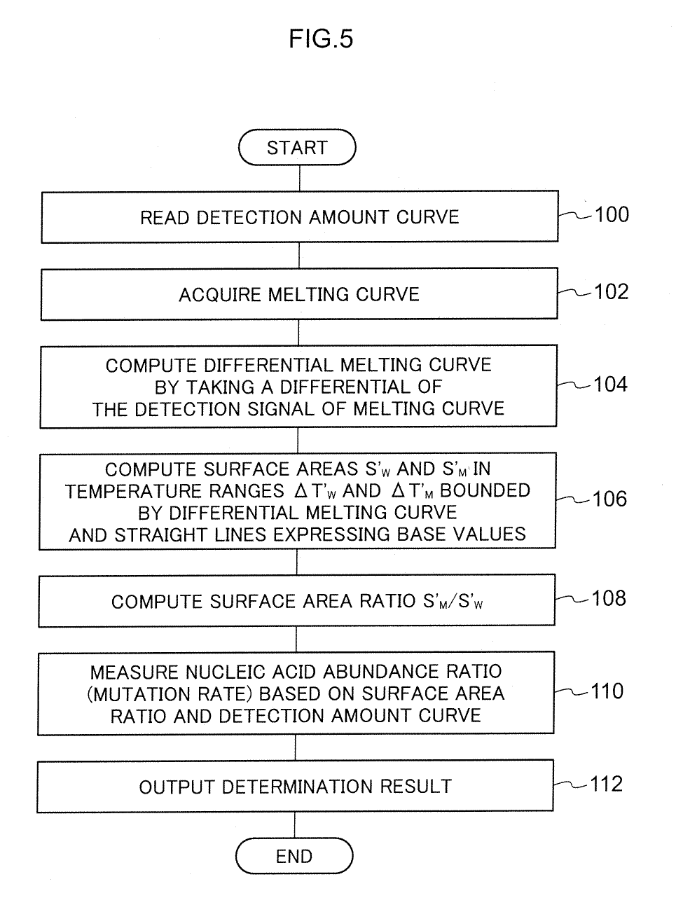

Explanation follows, with reference to FIG. 5, regarding a nucleic acid abundance ratio measurement routine executed by the nucleic acid presence ratio measurement device 10 of the present exemplary embodiment on a nucleic acid mixture to be measured (measurement target).

At step 100, the detection amount curve stored in the memory 26 is read in to the device 10.

Then, at step 102, melting curve data is acquired for a subject with unknown abundance ratio of two nucleic acids (the measurement target nucleic acid mixture). The melting curve data can, for example, be acquired from an external device, such as a melting curve analysis instrument connected through the network I/F 30, or be acquired by reading in data stored on a storage medium.

At step 104, the detection signal of the melting curve acquired at step 102 is differentiated with respect to temperature, and a differential melting curve is computed that expressed the relationship of the differential value of the detection signal against temperature.

At step 106, temperature ranges ΔT′_{W} and ΔT′_{M} are set for the differential melting curve computed at step 104, corresponding to the respective temperature ranges ΔT_{W} and ΔT_{M}, which have been set when the detection amount curve read in at step 100 was generated. The temperature ranges ΔT′_{W} and ΔT′_{M} may be set as the same temperature ranges as the temperature ranges ΔT_{W} and ΔT_{M}, which have been set when the detection amount curve was generated, or may be set as temperature ranges approximating to the temperature ranges ΔT_{W} and ΔT_{M}.

The surface area is then derived in each of the temperature ranges ΔT′_{W} and ΔT′_{M} for an area bounded by a straight line passing through the point corresponding to the lower limit and the point corresponding to the upper limit of the temperature range of the differential melting curve, and bounded by the differential melting curve itself. Specifically, the surface area S′_{W} for the temperature range ΔT′_{W} and the surface area S′_{M} for the temperature range ΔT′_{M} are computed according to Equation (1) and Equation (2) by a similar method as that employed to computing the surface area of each of the temperature ranges when the detection amount curve was generated.

At step 108, the surface area ratio S′_{M}/S′_{W} is computed with the surface areas S′_{W} and S′_{M} that have been computed at step 106.

At step 110, based on the surface area ratio S′_{M}/S′_{W} computed at step 108 and the detection amount curve read at step 100, the abundance ratio of nucleic acids in the sample is measured by computing the nucleic acid abundance ratio corresponding to the surface area ratio S′_{M}/S′_{W}. Thereby, the mutation rate in the sample is determined

Then, at step 112, the measurement result at step 110 is output by displaying the measurement result on the display section 14, and processing is completed.

In the above-described routine, the detection amount curve is read (at step 100) before the melting curve is obtained at step 102. However, embodiments are not limited to this, and the reading of the detection amount curve can be performed at any time before the abundance ratio of nucleic acids (the mutation rate) is measured based on the surface area ratio and the detection amount curve.

As explained above, according to the nucleic acid abundance ratio measurement device of the present exemplary embodiment, the abundance ratio of nucleic acids in a sample can be readily determined by simple computation based on: the detection amount curve, expressing the relationship between abundance ratios of two types of nucleic acid, and ratios of surface areas obtained from differential melting curves at two temperature ranges respectively containing the melting temperatures of each nucleic acid; and a surface area ratio obtained from a differential melting curve of a measurement target nucleic acid mixture of unknown abundance ratio at temperature ranges corresponding to the two temperature ranges of the differential melting curve. Thereby, the mutation rate in the sample can be readily determined.

### Example 1

Explanation follows regarding an Example of the present exemplary embodiment in which a c-Kit gene partial sequence is employed as the measurement target nucleic acid.

PCR and Tm analysis is performed on a sample of nucleic acid mixtures (at 10³ copies/reaction liquid) as shown in Table 3, with a full automatic SNP analyzer (product name: i-densy®; produced by Arkray Inc.). The composition of the PCR reaction liquid is shown in Table 1 and the conditions of the PCR and Tm analysis is shown in Table 4.

TABLE 1
In 50 μL of PCR Reaction Liquid Composition:

| | |
|---|---|
| 1 × | reaction buffer |
| 1.25 | U Taq polymerase |
| 1.5 | mmol/L MgCl₂ |
| 0.2 | mmol/L dNTP |
| 1 | μmol/L F-primer* |
| 0.5 | μmol/L R-primer* |
| 0.2 | μmol/L probe* |

*Sequences of F-primer, R-primer and the probe are shown in Table 2 (SEQ ID 1-3)

TABLE 2

| Name | Sequence | Mer |
|---|---|---|
| F-primer | 5′-tgtattcacagagacttggca-3′ | 21 |
| R-primer | 5′-gagaatgggtactcacgtttc-3′ | 21 |
| Probe | 5′-gatagtctctggctagacc-(BODIPY FL)-3′ | 18 |

TABLE 3

| | Mixing Proportions of Each Plasmid | |
|---|---|---|
| Sample Name | Wt | Mt |
| Wt 100% | 100% | 0% |
| Mt 10% | 90% | 10% |
| Mt 20% | 80% | 20% |
| Mt 30% | 70% | 30% |
| Mt 40% | 60% | 40% |
| Mt 50% | 50% | 50% |
| Mt 60% | 40% | 60% |
| Mt 70% | 30% | 70% |
| Mt 80% | 20% | 80% |
| Mt 90% | 10% | 90% |
| Mt 100% | 0% | 100% |

Note
that in this Example, Wt plasmid = 100% and Mt plasmid = 100% are also regarded as nucleic acid mixtures.

For the plasmid, pT7 Blue T-vector (trade name, available through TaKaRa Bio Inc.) to which the sequences (Wt Sequence: SEQ ID4, Mt Sequence: SEQ ID5) shown in Table 4 are inserted into, and linearized using Eco R1, is used. The Wt Sequence and the Mt Sequence differ in the base shown in capitals. The plasmid inserted with the Wt Sequence is referred to as the Wt plasmid, and the plasmid inserted with the Mt Sequence is referred to as the Mt plasmid.

Wt Sequence:
cactatagtattaaaaagttagttttcactctttacaagttaaaatgaa
tttaaatggttttcttttctcctccaacctaatagtgtattcacagaga
cttggcagccagaaatatcctccttactcatggtcggatcacaaagatt
tgtgattttggtctagccagagAcatcaagaatgattctaattatgtgg
ttaaaggaaacgtgagtacccattctctgcttgacagtcctgcaaagga
tttttagtttcaactttcgataaaaattgtttcctgtgactttcataat
gtaaat
Mt Sequence:
cactatagtattaaaaagttagttttcactctttacaagttaaaatgaa
tttaaatggttttcttttctcctccaacctaatagtgtattcacagaga
cttggcagccagaaatatcctccttactcatggtcggatcacaaagatt
tgtgattttggtctagccagagTcatcaagaatgattctaattatgtgg
ttaaaggaaacgtgagtacccattctctgcttgacagtcctgcaaagga
tttttagtttcaactttcgataaaaattgtttcctgtgactttcataat
gtaaat

TABLE 4
Conditions of PCR and Tm Analysis

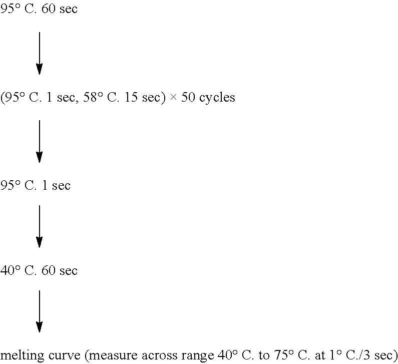

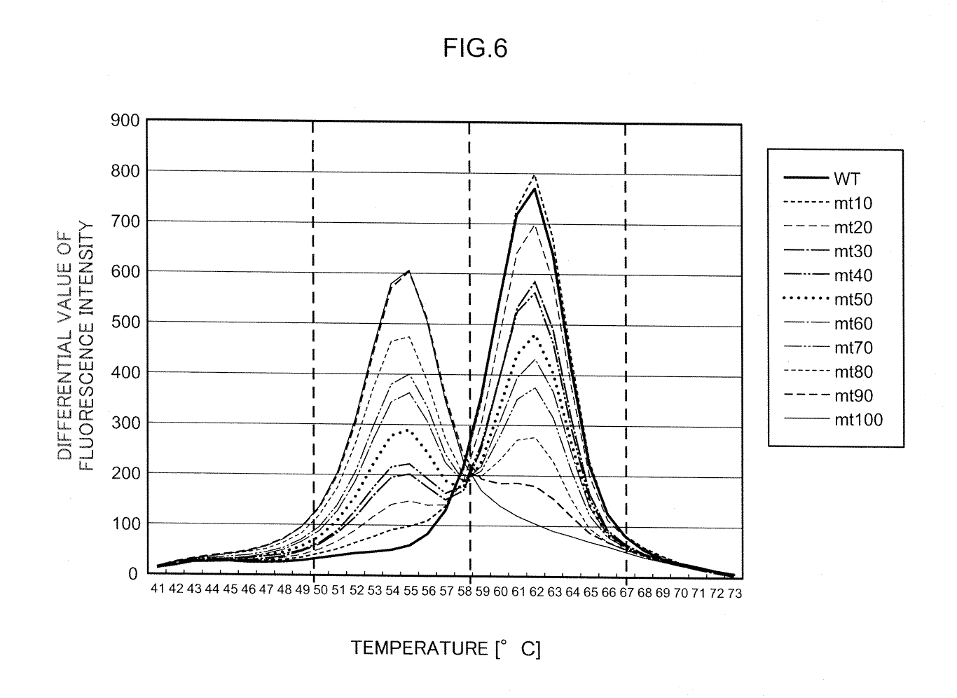

FIG. 6 illustrates differential melting curves of the samples measured according to the above conditions. In the present Example, fluorescence intensity is obtained as the detection signal. The peaks are extracted from the differential melting curves of FIG. 6, and the melting temperatures Tm_{W} and Tm_{M} are determined for each of the nucleic acids. The temperature range ΔT_{W} (58° C. to 66° C.) and the temperature range ΔT_{M} (50° C. to 58° C.) are then set so that the determined melting temperatures are respectively included therein. Then, the surface area S_{W} for the temperature range ΔT_{W} and the surface area S_{M} for the temperature range ΔT_{M} are derived according to Equation (1) and Equation (2), and the surface area ratio S_{M}/S_{W} is calculated. Table 5 illustrates the surface areas S_{M} and S_{W} and the surface area ratio S_{M}/S_{W} for each of the samples.

TABLE 5

| Sample Name | S_{M} | S_{W} | S_{M}/S_{W} |
|---|---|---|---|
| Wt 100% | 0 | 879 | 0.0% |
| Mt 10% | 0 | 924 | 0.0% |
| Mt 20% | 39.6 | 800.5 | 4.9% |
| Mt 30% | 121.9 | 652 | 18.7% |
| Mt 40% | 147.8 | 619 | 23.9% |
| Mt 50% | 238.5 | 509 | 46.9% |
| Mt 60% | 333.5 | 433.5 | 76.9% |
| Mt 70% | 375 | 346 | 108.4% |
| Mt 80% | 471 | 192 | 245.3% |
| Mt 90% | 618.5 | 43.5 | 1421.8% |
| Mt 100% | 632.5 | 0 | — |

A detection amount curve is generated based on the values shown in FIG. 5, with the proportion of Mt in the total sample shown on the horizontal axis and the surface area ratio S_{M}/S_{W} shown on the vertical axis. The generated detection amount curve here is the same as the example shown in FIG. 4.

This detection amount curve is employed to measure the nucleic acid abundance ratio of a measurement target nucleic acid mixture of unknown Wt and Mt abundance ratio.

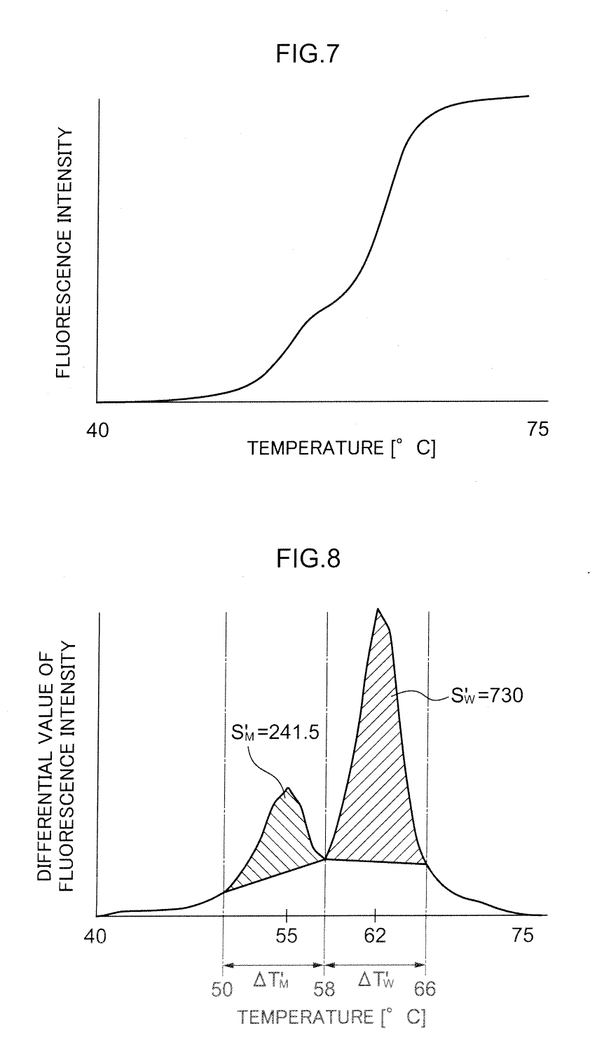

First, melting curve data, as illustrated in FIG. 7, expressing the relationship of fluorescence intensity against temperature of the measurement target nucleic acid mixture is acquired (step 102). Then, as shown in FIG. 8, differentials of the fluorescence intensities in the melting curve are taken, and a differential melting curve expressing the relationship of fluorescence intensity differentials against temperature is computed (step 104).

The temperature ranges ΔT′_{W} and ΔT′_{M} as the same temperature ranges as the temperature range ΔT_{W} 58° C. to 66° C.) and the temperature range ΔT_{M} (50° C. to 58° C.) which has been set when generating the detection amount curve of FIG. 4, are set for the computed differential melting curve. The surface area S′_{W} for the temperature range ΔT′_{W} and the surface area S′_{M} for the temperature range ΔT′_{M} are computed according to Equation (1) and Equation (2) (step 106). In this Example, S′_{W}=730 and S′_{M} =241.5 are obtained.

The surface area ratio S′_{M}/S′_{W} is based on the computed surface areas S′_{W} and S′_{M} (step 108). In this Example, S′_{M}/S′_{W}=0.331 (33.1%) is obtained.

The mutation rate is determined by computing the proportion of Mt, that is the mutation rate, corresponding to the surface area ratio S′_{M}/S′_{W} (=33.1%) (step 110). In this Example, the proportion of Mt is between 40 to 50%.

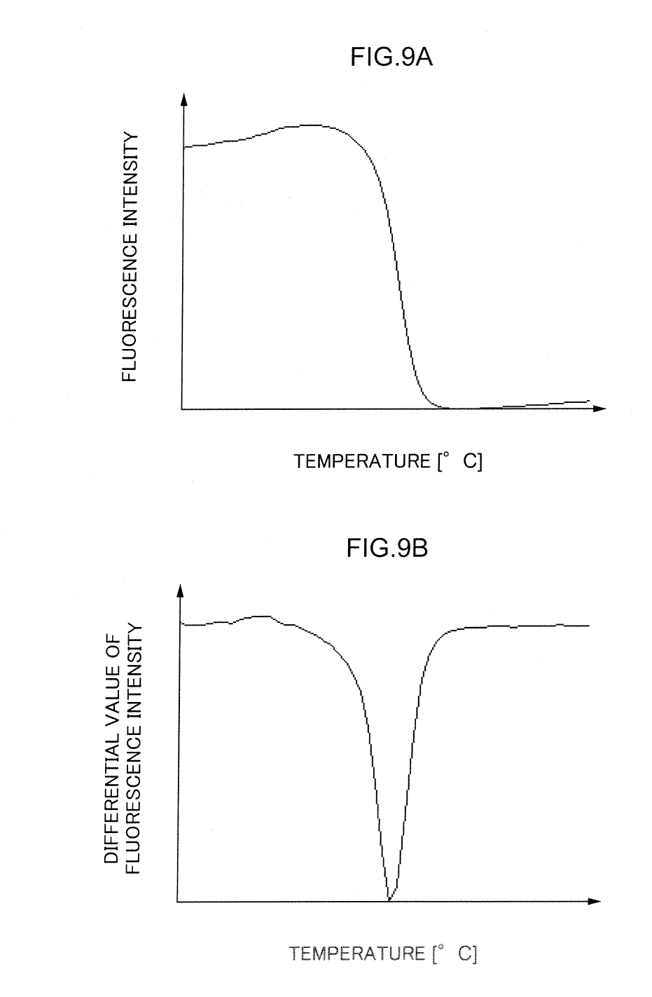

Explanation in the above Example is of a case in which a probe that emits fluorescent light is used, and the fluorescence intensity emitted in response to excitation light appropriate for the fluorescent dye is employed as the detection signal indicating the melting state. However, embodiments are not limited to this. For example, the degree of light absorption at 260 nm that increases due to dissociation of double-stranded nucleic acid may be employed as the detection signal. Further, explanation in the above Example is of a case in which a probe that quenches when double-strands are formed (not dissociated) is employed; however a probe may be employed that emits fluorescent light when double-strands are formed. In such cases, a melting curve as shown in FIG. 9A is obtained that expresses the relationship of fluorescence intensity against temperature. A differential melting curve such as shown in FIG. 9B is obtained therefrom.

Specific examples of fluorescent dyes include intercalaters such as ethidium bromide and SYBR® Green. Generally, in such fluorescent dyes, fluorescent light is emitted by double-strand formation, and the emission of fluorescent light is suppressed by double-strands dissociating. Fluorescent dyes may, for example, be conjugated to single-strand nucleic acid of at least one of the double-strands configuring the nucleic acid. Examples of single-strand nucleic acid conjugated with a fluorescent dye include a fluorescence quenching probe such as QPROBE® which is known as a guanine quenching probe used in the Example. A fluorescence quenching probe generally quenches fluorescence due to formation of double-strands and emits fluorescent light due to dissociating of the double-strands.

The detection signal representing the melting state of the nucleic acid mixture in the embodiments may be, for example, a signal generated due to non-melting of the sample and suppressed from generation due to melting of the sample as described above, or may be the opposite, a signal suppressed from generation due to non-melting of the sample and generated due to melting of the sample. The differential value of the detection signal may be, for example, a differential of the detection signal with respect to temperature expressed by (dF/dT), or may be expressed by (−dF/dT), where dF is the change in the detection signal and dT is the change in temperature. In a case in which detection signal generation is suppressed by melting of the sample, the peaks appear in valleys in the differential melting curve in which the detection signal differential values are expressed by (dF/dT), and the peaks appear in mountain shapes in the differential melting curve in which the detection signal differential values are expressed by (−dF/dT). In contrast, in cases in which the detection signal is generated by melting of the sample, the peaks appear in mountain shapes in the differential melting curve in which detection signal differential values are expressed by (dF/dT), and the peaks appear in valleys in the differential melting curve in which detection signal differential values are expressed by (−dF/dT). In both of these cases, the surface area of portions in specific temperature ranges bounded by the differential melting curve and a straight line representing base values can be derived. The differential value of the detection signal is not limited to a differential value of the detection signal differentiated with respect to temperature, and a differential value in which the detection signal is differentiated with respect to time may be employed.

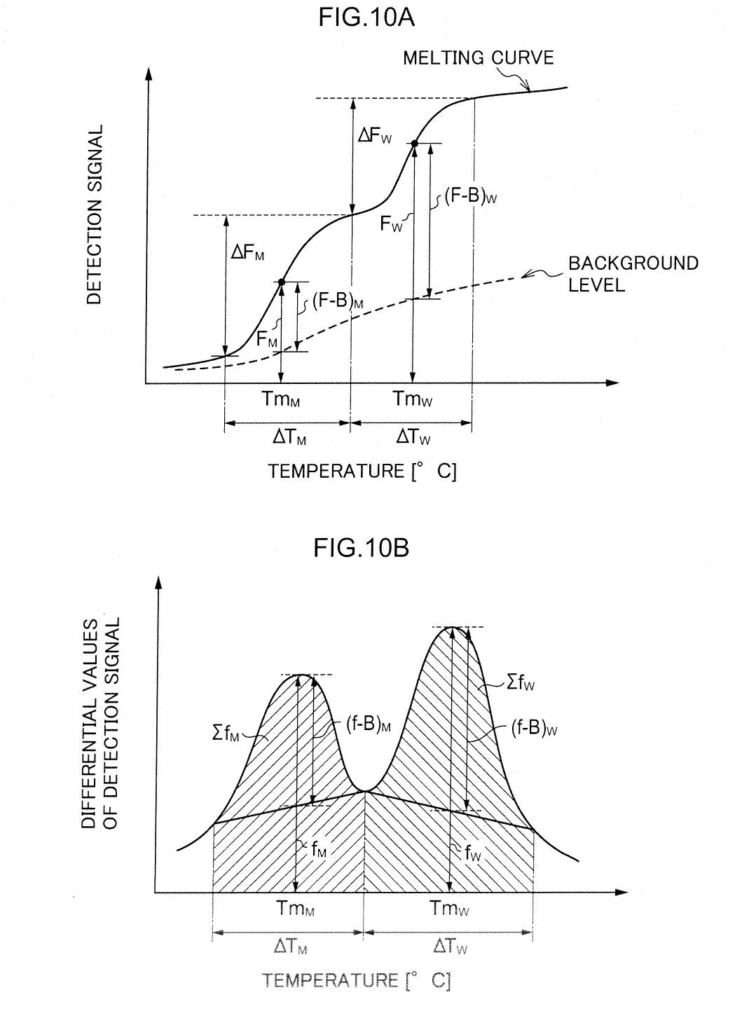

The above exemplary embodiment and the Example are explained using a case in which the surface area ratio is employed as a ratio of characteristic amounts which is obtained from the detection signal of the melting curve. However, embodiments are not limited to this. For example, as shown in FIG. 10A, a ratio (F_{M}/ F_{W}) which is the ratio of the detection signal level F_{M} at the melting temperature Tm_{M} to the detection signal level F_{W} at the melting temperature Tm_{W} may be employed as the ratio of characteristic amounts. Similarly, a ratio (ΔF_{M}/ΔF_{W}) which is the ratio of the detection signal level difference ΔF_{M} between the detection signal level at the lower limit of the temperature range ΔT_{M} and the detection signal level at the upper limit of the temperature range ΔT_{M} to the detection signal level difference ΔF_{W} between the detection signal level at the lower limit of the temperature range ΔT_{W} and the detection signal level at the upper limit of the temperature range ΔT_{W} may be employed as the ratio of characteristic amounts. Further, a ratio ((F−B)_{M}/(F−B)_{W}), which is the ratio of (F−B)_{M} that is the value of the detection signal level at the melting temperature Tm_{M} from which the background level thereof has been subtracted to the value of (F−B)_{W} that is the detection signal level at the melting temperature Tm_{W} from which the background level thereof has been subtracted, may be employed as the ratio of characteristic amounts.

Further, as shown in FIG. 10B, a ratio (f_{M}/f_{W}) which is the ratio of the detection signal differential value f_{M} at the melting temperature Tm_{M} to the detection signal differential value f_{W} at the melting temperature Tm_{W} may be employed as the ratio of characteristic amounts. Similarly, a ratio (Σf_{M}/Σf_{W}) which is the ratio of Σf_{M}, that is the sum of differential values of detection signal over the temperature range ΔT_{M} (corresponding to the surface area of the shaded portion slanting up to the right of FIG. 10B), to Σf_{W}, that is the sum of differential values of detection signal over the temperature range ΔT_{W} (corresponding to the surface area of the shaded portion slanting down to the right of FIG. 10B), may be employed as the ratio of characteristic amounts. Similarly, a ratio ((f−B)_{M}/(f−B)_{W}) which is the ratio of (f−B)_{M}, that is the value of the detection signal differential value at the melting temperature Tm_{M} from which the background level thereof has been subtracted, to (f−B)_{W}, that is the value of the detection signal differential value at the melting temperature Tm_{W} from which the background level thereof has been subtracted, may be employed as the ratio of characteristic amounts. (f−B)_{W} can be derived by subtracting from f_{W} a value corresponding to the melting temperature Tm_{W} on a straight line passing through points corresponding to the lower limit and upper limit values of the temperature range ΔT_{W}. Similarly, (f−B)_{M} can be derived by subtracting from f_{M} a value corresponding to the melting temperature Tm_{M} on a straight line passing through points corresponding to the lower limit and upper limit values of the temperature range ΔT_{M}.

In the above exemplary embodiment, measurement is made of an abundance ratio between nucleic acids that differ by only a single base. However, embodiments are not limited to the nucleic acids being homologous, and application may be made even to completely non-homologous nucleic acids as long as they have different melting temperatures from each other. For example, an abundance ratio can be determined between a gene A and a gene B for a nucleic acid mixture of non-homologous gene A and gene B having differing melting temperatures.

Whereas in the above exemplary embodiment, detection has been made by employing the same probe for each of the nucleic acids, different probes may be employed for each of the nucleic acids.

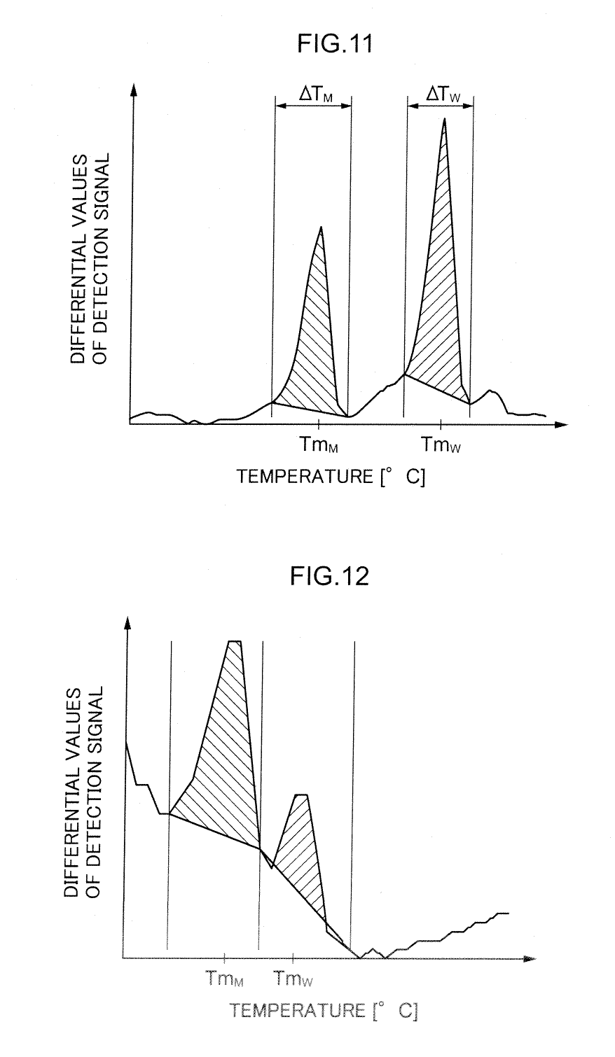

Further, in the above exemplary embodiment and Example, explanation is given of cases in which the temperature ranges respectively containing the melting temperatures of the nucleic acids (ΔT_{W} and ΔT_{M}) are contiguous. However, as shown in FIG. 11, non-contiguous ranges can be set for the temperature ranges when the respective melting temperatures are separated from each other.

In the above exemplary embodiment and Example, explanation is given of cases in which base values for subtracting the background level are taken as values on a line passing through points corresponding to the lower limit and the upper limit of the temperature range in the differential melting curve. However, for example, a fixed value may be employed as the base value, such as the differential value of the detection signal corresponding to either the upper limit value or the lower limit value of the temperature range. Nonetheless, as shown in FIG. 12, when there is a considerably difference between the detection signal differential values corresponding to the lower limit and upper limit values of the temperature range (when a shift in the base values of a specific amount or greater occurs), influence from the background can be removed with better precision by adopting the method of taking values on a straight line passing through points corresponding to the lower limit and the upper limit of the temperature range in the differential melting curve as the base values, as in the above exemplary embodiment and Example. Further, the base values may be determined on a curve instead of a straight line.

Explanation is given in the above exemplary embodiment and Example of cases in which a nucleic acid abundance ratio is computed for nucleic acid mixtures of different abundance ratios of two types of nucleic acid. However, the present invention can be similarly applied to nucleic acid mixtures of different abundance ratios of three or more types of nucleic acid.

The above exemplary embodiment and Example describes cases in which a detection amount curve such as shown in FIG. 4 is employed as the detection amount data. However, embodiments are not limited thereto, and the detection amount data in which the relationship between a ratio of characteristic amounts and the abundance ratio of nucleic acids is expressed in a tabular format or in a computation formula may be employed.

In the above exemplary embodiment, the measurement result is displayed on the display section 14. However, embodiments are not limited thereto, and a printer may be provided and the measurement result may be printed out on a medium such as paper, the measurement result may be recorded on a portable recording medium, or may be output to an external device connected through the I/O port 28 and the network I/F 30.

The nucleic acid abundance ratio measurement device of the present exemplary embodiment may be further provided with a determination section that determines progression of an illness of a patient based on a relationship between illness progression and nucleic acid abundance ratios that has been determined in advance, and on the computed nucleic acid abundance ratio. The abundance ratio of mutated genes and normal genes may be measured and employed as a parameter that is considered when looking into a patient's condition. For example, a table may be generated in advance that defines relationship between proportions of mutated genes (for example genes related to malignant or cancer conditions) to normal genes in nucleic acids isolated from blood, and illness progression. The abundance ratio of mutant genes to normal genes of a patient may then be measured, and the illness progression can be assessed by comparison to the table generated in advance. Monitoring of a patient's condition, such as illness progression, can accordingly be performed by employing the determined nucleic acid abundance ratio.

The nucleic acid abundance ratio measurement device of the present exemplary embodiment may be further provided with a determination section that determines at least one of constitution of a patient and/or drug administration amount appropriate for the constitution. This determination is based on a predetermined relationship between the computed nucleic acid abundance ratio and constitution and/or drug administration amount appropriate for the constitution, and based on the computed nucleic acid abundance ratio of a patient. Constitution relates to the ease with which an investigation subject succumbs to a particular disease, and the effectiveness of a particular drug administration dose. For example, a table is generated in advance of a relationships between the proportion of a target gene of known copy number in a given gene (a gene with copy number variance), and factors as the ease of succumbing to a particular disease, the presence or absence of a particular disease, medical condition, drug effectiveness, drug administration amount and the like. The abundance ratio of the target gene in the gene with known copy number variance is determined and compared with the table that has been generated in advance. Thereby, the constitution, such as ease of succumbing to a particular disease and drug effectiveness, the presence or absence of a disease, medical condition, and appropriate drug administration amount can be determined. In this way, the determined abundance ratio can be employed to determine the constitution of an investigation subject and drug administration amount thereto. For example, subjects with a high copy number of CCL3L are known to have a constitution not readily susceptible to HIV, and subjects with a low copy number of FCGR3B are known to have a constitution readily susceptible to autoimmune diseases, such as systemic lupus erythematosus. Genes associated with conditions such as autism, schizophrenia, and sudden learning disabilities are also known to be expressed in association with high copy numbers.

Further, an embodiment may be configured as a nucleic acid abundance ratio measurement kit including a probe that can be hybridized with an area in a nucleic acid sequence that includes a target mutation, and a set of primers that can amplify the nucleic acid sequence including the target mutation. Using a measurement kit including such reagents may be advantageous in that target mutations of genes can be more easily detected and the abundance can be more easily measured.

Sequences of the probe and the primers that configure the nucleic acid abundance ratio measurement kit can be appropriately designed by a person skilled in the art, based on the sequence of the target gene and the sequence of the target mutated gene, if known. The length of the probe and annealing positions of the set of primers that are necessary to effectively implement testing can also be appropriately adjusted by a person skilled in the art.

It is preferable for the probe to be a labeled probe that is labeled from the standpoint of the efficiency of the testing. Specific examples of labeling material for the labeled probe include fluorescent dyes and fluorophores. A specific example of the labeled probe may preferably be a probe that has been labeled by a fluorescent dye and emits fluorescence independently, and in which the fluorescence emission decreases (quenches, for example) due to formation of a hybrid.

Probes using such a quenching phenomenon are generally referred to as fluorescence quenching probes. Among them, a probe in which a base in the 3′ region (3′-end) or the 5′ region (5′-end) has been labeled by a fluorescent dye, and the labeled base is cytosine (C) is preferable. In this case, it is preferable to design the base sequence of the labeled probe such that a base in the testing target sequence which is to be paired with the terminal base (C) of the labeled probe, or a base that is separated by one to three bases from the base in the testing target sequence which is to be paired with the terminal base (C) of the labeled probe, is guanine (G). This kind of probe is generally referred to as a guanine quenching probe, which is known as a Q-Probe. When such a guanine quenching probe hybridizes with the testing target sequence, it exhibits a phenomenon such that fluorescence emission of the fluorescent dye is weakened (i.e., the intensity of the fluorescence decreases) due to the terminal base C which has been labeled by the fluorescent dye approaching the base G in the testing target sequence. By using such a probe, hybridization and disaggregation in the testing target sequence can be easily confirmed by variations in signals indicating the fluorescence emission. The labeling material can usually be conjugated to phosphate groups in a nucleotide.

Reagents included in the nucleic acid abundance ratio measurement kit of the present embodiment can be kept respectively in different containers, or be kept in the same container. The term “different containers” herein may be a container which is partitioned such that the reagents are maintained in non-contact states, and may not necessarily be separate containers that can be respectively handled independently.

The nucleic acid abundance ratio measurement kit of the present embodiment may further contain reagents or buffers necessary for amplification such as polymerase, reagents or buffers necessary for hybridization, diluents for diluting a testing subject, and the like. It is preferable for the nucleic acid abundance ratio measurement kit of the present embodiment to further contain an explanatory leaflet that explains the nucleic acid abundance ratio measurement method, an instruction leaflet regarding reagents that are included or can be additionally included in the kit, and the like.

Explanation in the above exemplary embodiments are given for cases in which melting curve data is acquired from an external instrument such as a melting curve analyzer, or acquired by reading data stored on a storage medium. However, the melting curve analyzing device may be integrated together with the nucleic acid abundance ratio measurement device of the present exemplary embodiment. Specifically, nucleic acid abundance ratio measurement device may include a temperature controller that controls temperature change of the nucleic acid mixture, and a measurement section for measuring a detection signal such as degree of light absorption, fluorescence intensity, and relative fluorescence, in addition to the configuration described in the above exemplary embodiments. Data of the melting curve may be obtained by the measurement section by measuring the detection signal while the temperature of the nucleic acid mixture of the test sample is being changed by the temperature controller. The nucleic acid abundance ratio of the sample can then subsequently be determined in the same manner to that of the above exemplary embodiments.

The above nucleic acid abundance ratio measurement routine may be provided via a storage medium storing a program thereof.

What is claimed is:

1. A nucleic acid abundance ratio measurement device comprising:
a detection section that detects a detection signal over different temperature ranges of a melting curve for a nucleic acid mixture having one or more melting temperatures; and
an abundance ratio computation section that computes a nucleic acid abundance ratio based on a ratio of characteristic amounts obtained from the detection signal detected by the detection section and based on detection amount data.

2. The device of claim 1, further comprising:
a storage section that stores the detection amount data that, based on respective melting curves each expressing a relationship between temperature and a detection signal obtained from each of a plurality of nucleic acid mixtures of different abundance ratios of nucleic acids having different melting temperatures, expresses a relationship between ratios of characteristic amounts obtained from the detection signal of the melting curves over a plurality of temperature ranges respectively containing the melting temperatures, and the abundance ratios; or
an input section that inputs the detection amount data.

3. The device of claim 1, wherein the characteristic amount ratio is set as one of:
a ratio of a first level of the detection signal level substantially at a first melting temperature from which a background level thereof has been subtracted, and a second level of the detection signal substantially at a second melting temperature from which a background level thereof has been subtracted;
a ratio of a first differential value of the detection signal level substantially at a first melting temperature from which a background level thereof has been subtracted and a second differential value of the detection signal level substantially at a second melting temperature from which a background level thereof has been subtracted;
a ratio of a first sum of differential values of the detection signal over a first temperature range and a second sum of differential values of the detection signal over a second temperature range; or
a ratio of a first sum of differential values of the detection signal over a first temperature range from which a sum of background levels thereof has been subtracted, and a second sum of differential values of the detection signal over a second temperature range from which a sum of background levels thereof has been subtracted.

4. The device of claim 1, wherein the characteristic amount ratio is set as a ratio, in a second melting curve expressing a relationship between temperature and differential values of the detection signal, of a first surface area of a region bounded by a straight line passing through a point corresponding to a lower limit value of a first temperature range and a point corresponding to an upper limit value of the first temperature range and bounded by the melting curve, and a second surface area of a region bounded by a straight line passing through a point corresponding to a lower limit value of a second temperature range and a point corresponding to an upper limit value of the second temperature range and bounded by the melting curve.

5. The device of claim 4, wherein the lower limit values or the upper limit values of the first and second temperature ranges are selected from:
temperatures at which the differential value takes a minimum value between two peaks of the second melting curve,
temperatures corresponding to tails of the peak of the second melting curve, or
temperatures within ±15° C. from a temperature corresponding to the peak of the second melting curve.

6. The device of claim 4, wherein the lower limit values or the upper limit values of the first and second temperature ranges are temperatures within ±10° C. from a temperature corresponding to the peak of the second melting curve.

7. The device of claim 4, wherein the lower limit values or the upper limit values of the first and second temperature ranges are temperatures within ±7° C. from a temperature corresponding to the peak of the second melting curve.

8. The device of claim 4, wherein the upper limit value of one of the first or second temperature range is different from the lower limit value of the other of the first or second temperature range that is a higher range than that of the one of the first or second temperature range.

9. The device of claim 4, wherein a width from the lower limit value to the upper limit value of the first temperature range and a width from the lower limit value to the upper limit value of the second temperature range are identical.

10. The device of claim 4, wherein a width from the lower limit value to the upper limit value of the first temperature range and a width from the lower limit value to the upper limit value of the second temperature range are different.

11. The device of claim 1, further comprising:
a temperature controller that controls change in temperature of the nucleic acid mixture; and
a measurement section that measures degree of light absorption, fluorescence intensity or relative fluorescence intensity as the detection signal.

12. A nucleic acid abundance ratio measurement method comprising:
detecting a detection signal over different temperature ranges for a melting curve of a nucleic acid mixture having one or more melting temperatures; and
computing a nucleic acid abundance ratio based on a ratio of characteristic amounts obtained from the detection signal that has been detected and based on detection amount data.

13. A nucleic acid abundance ratio measurement method comprising:
generating detection amount data based on melting curves each expressing a relationship between temperature and a detection signal obtained from each of a plurality of nucleic acid mixtures of different abundance ratios of nucleic acids having different respective melting temperatures, the detection amount data expressing a relationship between ratios of characteristic amounts obtained from the detection signal of the melting curves over a plurality of temperature ranges respectively containing the melting temperatures, and the abundance ratios;
computing a ratio of characteristic amounts based on a detection signal of a melting curve of a measurement target nucleic acid mixture over temperature ranges corresponding to each of the plurality of respective temperature ranges; and
computing a nucleic acid abundance ratio based on the computed ratio of characteristic amounts and the generated detection amount data.

14. A non-transitory storage medium storing a program that causes a computer to execute abundance ratio measurement processing, the abundance ratio measurement processing comprising:
receiving a detection signal of a melting curve of a nucleic acid mixture leaving one or more melting temperatures, the detection signal being detected over different temperature ranges; and
computing a nucleic acid abundance ratio based on a ratio of characteristic amounts obtained from the received detection signal and based on detection amount data.

15. A non-transitory storage medium storing a program that causes a computer to execute abundance ratio measurement processing, the abundance ratio measurement processing comprising:
acquiring detection amount data from a storage section that stores the detection amount data that, based on respective melting curves each expressing a relationship between temperature and a detection signal obtained for each of a plurality of nucleic acid mixtures of different abundance ratios of nucleic acids having different respective melting temperatures, expresses a relationship between ratios of characteristic amounts obtained from the detection signals of the melting curves over a plurality of temperature ranges respectively containing the melting temperatures, and respective abundance ratios, or acquiring the detection amount data input by an input section;
computing a ratio of characteristic amounts based on a detection signal of a melting curve of a measurement target nucleic acid mixture, the detection signal detected over temperature ranges respectively corresponding to each of the plurality of temperature ranges; and
computing a nucleic acid abundance ratio based on the computed ratio of characteristic amounts and the acquired detection amount data.

16. A determination method of determining a condition of a patient based on:
a nucleic acid abundance ratio obtained with the nucleic acid abundance ratio measurement device of claim 1; and
a relationship determined in advance between nucleic acid abundance ratios and patient condition.

17. A determination method of determining a constitution of a patient and/or an appropriate drug administration amount for the constitution based on:
a nucleic acid abundance ratio obtained with the nucleic acid abundance ratio measurement device of claim 1; and
a relationship determined in advance between nucleic acid abundance ratios and constitutions and/or appropriate drug administration amount for the constitutions.

18. A nucleic acid abundance ratio measurement kit that is used in the measurement of a nucleic acid abundance ratio with the nucleic acid abundance ratio measurement device of claim 1, the kit comprising:
a probe that can be hybridized with a region of a nucleic acid sequence that includes a target mutation that may exist in the nucleic acid mixture; and
a set of primers that can amplify the nucleic acid sequence that includes the target mutation.

19. The nucleic acid abundance ratio measurement kit of claim 18, wherein the probe is a labeled probe.


SEQUENCE LISTING (5 sequences)

SEQ ID NO: 1 - DNA, 21 bp, Artificial Sequence
  primer_F
tgtattcacagagacttggca

SEQ ID NO: 2 - DNA, 21 bp, Artificial Sequence
  Primer_R
gagaatgggtactcacgtttc

SEQ ID NO: 3 - DNA, 18 bp, Artificial Sequence
  Probe
gatgtctctggctagacc

SEQ ID NO: 4 - DNA, 300 bp, Artificial Sequence
  Wt Sequence
cactatagtattaaaaagttagttttcactctttacaagttaaaatgaatttaaatggttttcttttctcctccaacctaatagtgtattcacagagacttggcagccagaaatatcctccttactcatggtcggatcacaaagatttgtgattttggtctagccagagacatcaagaatgattctaattatgtggttaaaggaaacgtgagtacccattctctgcttgacagtcctgcaaaggatttttagtttcaactttcgataaaaattgtttcctgtgactttcataatgtaaat

SEQ ID NO: 5 - DNA, 300 bp, Artificial Sequence
  Mt Sequence
cactatagtattaaaaagttagttttcactctttacaagttaaaatgaatttaaatggttttcttttctcctccaacctaatagtgtattcacagagacttggcagccagaaatatcctccttactcatggtcggatcacaaagatttgtgattttggtctagccagagtcatcaagaatgattctaattatgtggttaaaggaaacgtgagtacccattctctgcttgacagtcctgcaaaggatttttagtttcaactttcgataaaaattgtttcctgtgactttcataatgtaaat